(19) United States
(12) Patent Application Publication
(10) Pub. No.: US 2008/0306361 A1
(43) Pub. Date: Dec. 11, 2008

(54) Optical screening device

(76) Inventor: Joshua Friedman, Ridgefield, CT (US)

Correspondence Address: Eugene Lieberstein, 2151 Long Ridge Road, Stamford, CT, 06903, US

(21) Appl. No.: 11/811,580

(22) Filed: Jun. 11, 2007

(51) Int. Cl. A61B 6/00

(52) U.S. Cl. 600/306

(57) ABSTRACT

An optical screening device for detecting abnormal skin tissue indicative of disease including a handle having an elongated stem adapted for holding the optical screening device in one hand and a head for housing an array of light emitting diodes with each diode in the array being spaced apart to form a given geometry with each diode selected to emit light in a wavelength range between 340 nm to 470 nm; a high pass filter supported by said handle in close proximity to said array of light emitting diodes with the filter being of a size to provide a defined viewable area for visibly detecting irradiated light reflected from skin tissue under observation and adapted to block light at least below 380 nm; and a source of electrical power for energizing said array of light emitting diodes.

### FIELD OF INVENTION

This invention relates to an optical screening device for detecting abnormal skin tissue in a mammal and particularly in the mouth indicative of disease such as cancer or a pre-cancerous skin lesion.

### BACKGROUND

Oral cancer affects over 30,000 people today in the U.S. alone. The treatment for this disease is most effective when diagnosed early. When diagnosed late the treatment is unpleasant, disfiguring and often not effective. Advanced oral cancer is treated with surgery, chemo and radiation much the same as other aggressive cancers.

The early signs of oral cancer are not always discernable upon visual examination. In an attempt to improve the visualization other modalities have been developed to assist in screening for cancer and pre-cancerous lesions. Some of these procedures involve the use of toluidine blue rinse, acetic acid rinse and biofluorescence.

As in all cancers, a biopsy is used to make a positive diagnosis; however the process of taking a biopsy tissue sample is time consuming, costly and painful. It also requires special training and as consequence, most general dentists elect to send patients to an oral surgeon or oral pathologist for a biopsy procedure.

It would be highly desirable, therefore, to have a screening device that is easy to use, low in cost and effective. Such a device is useful even if a biopsy is still necessary provided it could eliminate false positives, permit the unnecessary taking of a biopsy or be indicative of when a biopsy for lesions is likely to be cancer or pre-cancer. Such a device would also save time, money and lives.

In the past, chemoluminesence together with an acetic rinse have been used to detect acetowhite lesion or luekelakia. This technique has been used in the screening for cervical cancer as well as oral cancer. However, it does not work for all lesions and does not delineate the border between healthy and pre-cancerous or dysplastic or cancerous tissue.

It is also known to use bioflouresence for detection of pre-oral and oral cancer. This technique for pre-oral and oral cancer detection uses a light source that can typically provide energy in the 300 NM to 470 NM range. It is known to those skilled in the art that within this wavelength distribution, oral tissue will fluoresce, while diseased tissue that exhibits varying degrees of dysplasia (pre cancer) will not fluoresce. It is also known that fluorescence normally takes place at a wavelength 40-60 NM higher than the excitation source or energy. In order to enhance the contrast between healthy and diseased tissue, a filter can be employed to block the excitation source and pass the fluorescent energy.

A device designed to use the technique of bioflouresence for oral cancer screening is currently manufactured by LED Electronics, Vancouver Canada. This device is described in detail in patent application Ser. No. 11/016,567, publication number US 2005/0234526 A1, by Gilhuly and Whitehead and uses a metal halide light source with a fiber optic or liquid light guide together with associated optics and filters. The device described in the Gilhuly patent application has many drawbacks based upon its use of a metal halide lamp, specialized power supply, bandpass filter, means to block excess heat and unwanted energy, fiber optic light guide and specialized means to view the tissue. A metal halide lamp produces a broad range or energy spectrum from below 300 NM to visible light and infra-red energy well above 800 NM. Accordingly, various blocking filters are required to provide useful excitation energy in the required spectrum. Moreover, since a metal halide light source provides most of its energy outside the useful wavelength spectrum for this procedure, this device is complex, unnecessarily large, bulky and extremely expensive.

### SUMMARY OF THE INVENTION

The optical screening device of this invention employs an array of light emitting diodes and a single blocking filter to cause suspected skin tissue to fluoresce enabling the discrimination between cancer, pre-cancerous and normal tissue. The array of light emitting diodes may consist of only one interconnected array of light emitting diodes to emit light in a wavelength range of between 340 nm to 370 nm or between 420 nm to 450 nm or may include a first and second array of interconnected light emitting diodes to emit light in a first wavelength range of between 340 nm to 370 nm and in a second wavelength range of between 420 nm to 450 nm. In the latter case it is preferred to also include switching means to activate either the first plurality of interconnected light emitting diodes or the second plurality of interconnected light emitting diodes. The blocking filter provides a screening area for visually observing the suspected skin tissue and should be of a defined size.

### BRIEF DESCRIPTION OF THE DRAWINGS

FIG. 1 is a front view of the device of the present invention showing the array of LED's in the head of the device and showing with dotted lines the internal batteries as the power source for the device;

FIG. 2 is a schematic view in perspective of the device in operation for screening the oral cavity in the mouth of a dental patient;

FIG. 3 is a circuit schematic of one embodiment of a control circuit and voltage regulator for activating the array of LED's in the device of the present invention;

FIG. 4 is a circuit schematic of a second embodiment of a control circuit and voltage regulator for activating the array of LED's in the device of the present invention; and

FIG. 5 is a partial view of an alternate embodiment of the present invention having a dual array of LED's in the head of the device.

### DETAILED DESCRIPTION

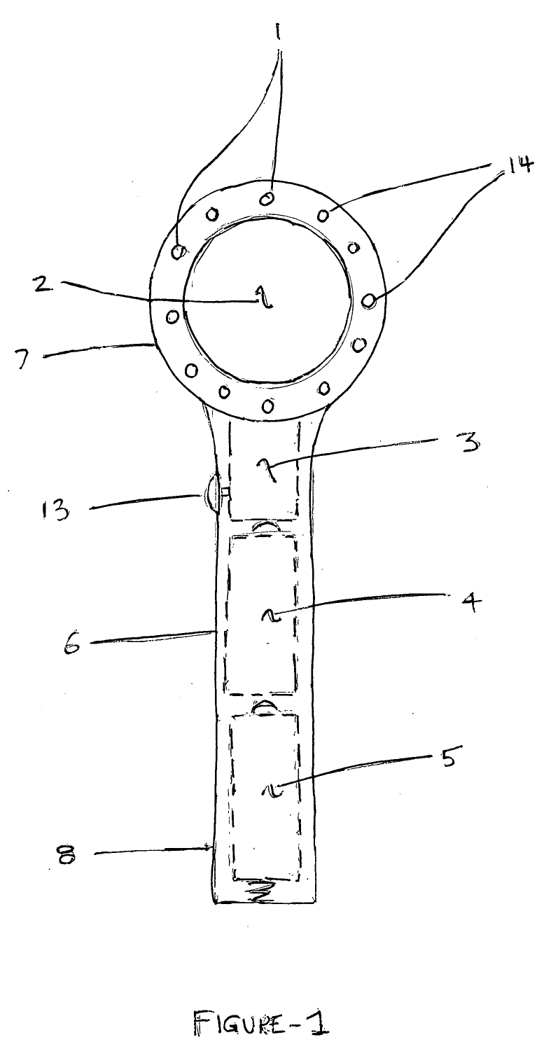

The optical screening device of the present invention is illustrated in FIGS. 1-4 represented by a simple hand held device 8 including a handle 6 and a head 7 extending from the handle 6. The head 7 defines a housing in which an array of light emitting diode's (LED's) 1 is mounted. A power source 4 or 5 for the array of light emitting diode's (LED's) 1, which may include a battery B, is preferably located in the handle 6. Light emitting diodes are low in cost and have lower power requirements due to their efficiency. A single filter 2, preferably a high pass filter as is known to those skilled in the art or a bandpass filter, is supported by the device 8 at a location adjacent to the array of LED's 1 so that a substantial amount of the irradiated light reflected from skin tissue to be observed by the screening device 8 will primarily pass through the filter 2. Thus the filter simultaneously acts as a viewing area for the optical sensing device 8 as well as to block unwanted light energy of the primary excitation.

The array of LED's 1 are interconnected to one another to emit light in a wavelength range of between 340 NM to 470 NM when connected to a power source. The array of LED's 1 may be arranged to form any desired geometry but should preferably surround the filter 2 so that the area circumscribing the filter defines a fixed viewable window area for the screening device 8. The array of LED's 1 produce energy in a desired wavelength to provide a bioflourescent effect without the need for special power supplies, bandpass filters, heat blocking filters or light guides as in the prior art device.

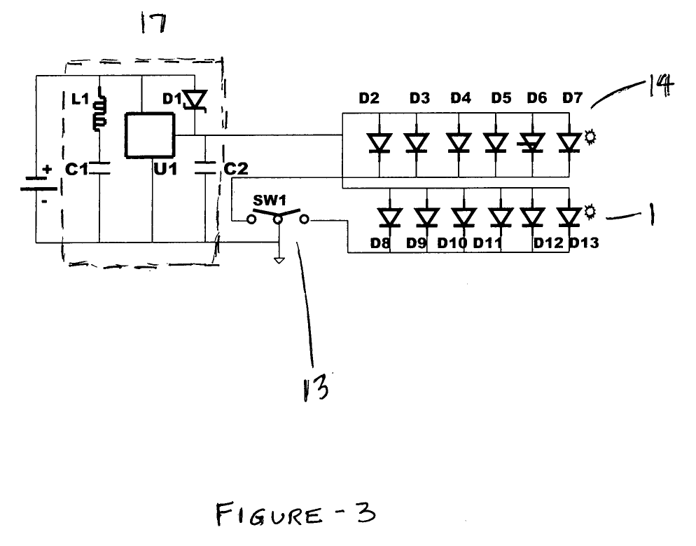

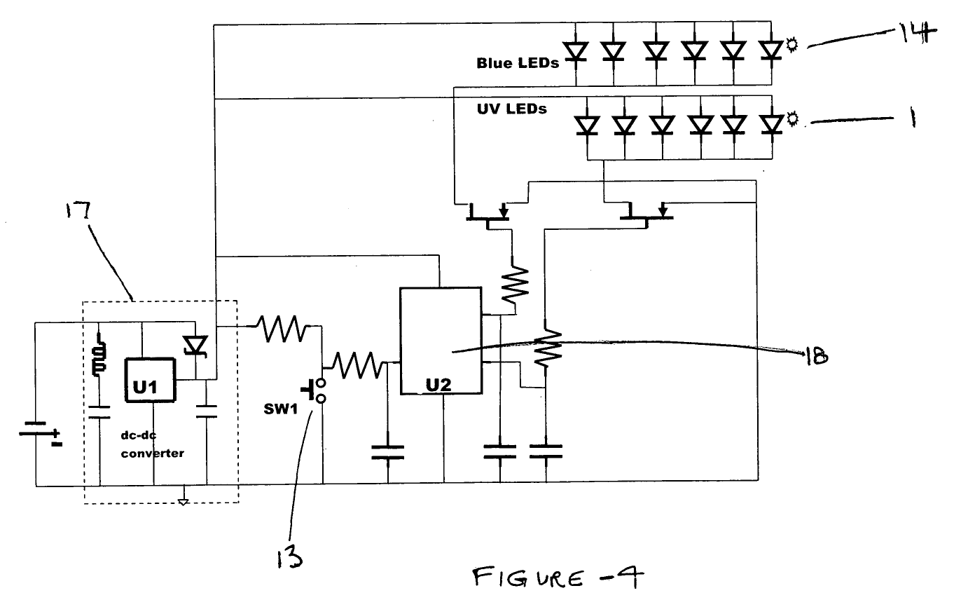

The power source for the array of LED's 1 can be a conventional power source such as a battery or plural batteries 4 and 5 as shown in FIG. 1 which may be connected to a control circuit 3 as shown in FIG. 3 or 4 mounted in the handle part 6 of the device. The power source may also be rechargeable. The handle 6 and head 7 may be composed of metal or of plastic.

In order to maintain constant light over a wide range of battery voltage, a voltage regulator circuit 17 may be included in the control circuit 3 for regulating the voltage of the power source.

### Device Operation

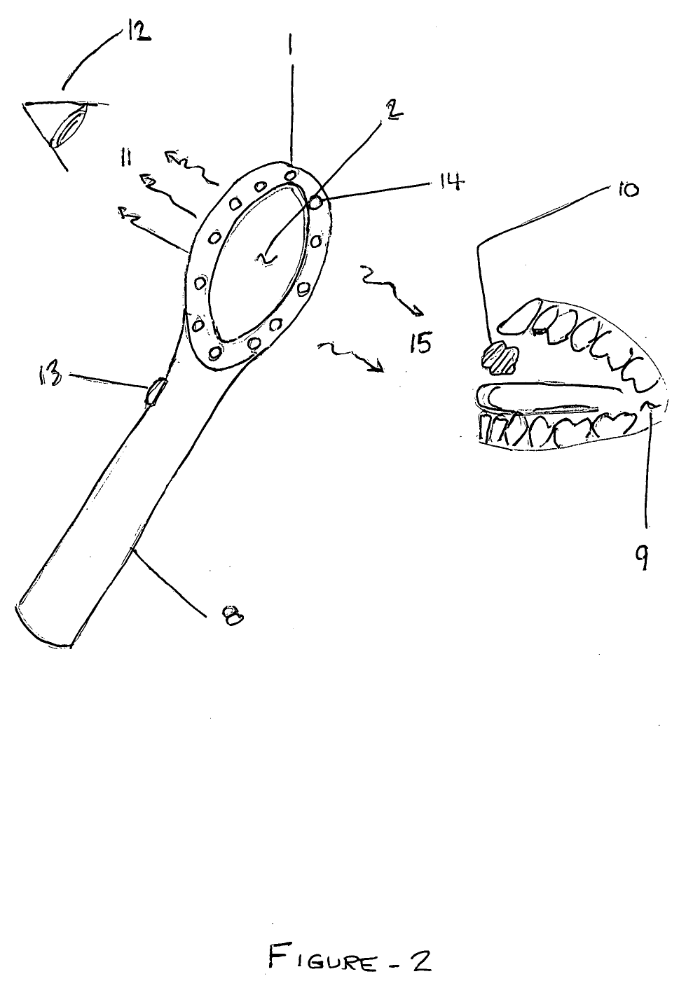

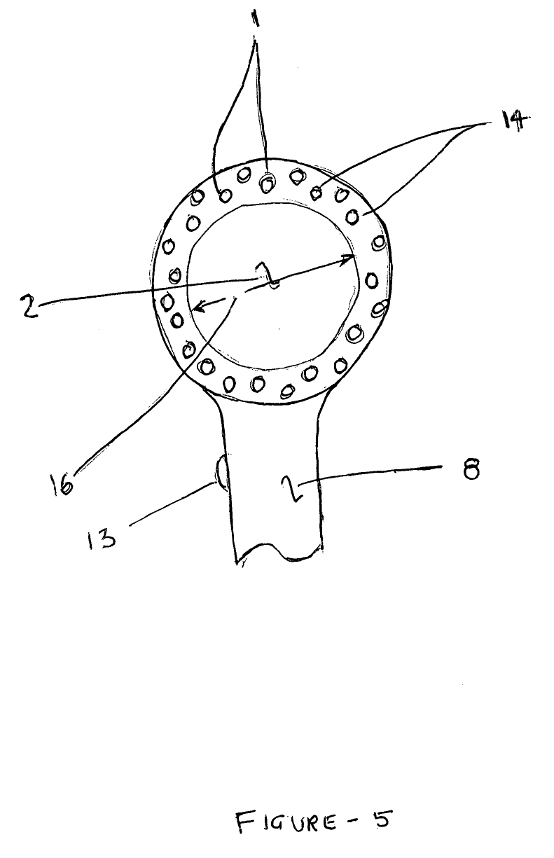

As shown in FIG. 2 the LED's 1 irradiate the mouth and associated oral structures which include the palate, tongue and oral mucosa 9. Irradiation may first be done with an array of LED's generating energy in the 340-370 NM range. A second examination using another or second array of LED's may then be made with energy in the 420-450 NM range. This can be accomplished with two devices 8 or using a single device 8 having a first and second array of LED's 1 mounted preferably in a concentric arrangement surrounding a filter 2 with each array of LED's consisting preferably of from 6 to 12 LED's as shown in FIG. 5. In the latter case a switch 13 is placed in the 340-370 NM mode and LED's 1 will illuminate 15 the suspected tissue 10. Next, a switch 13 is place in the 420-450 NM mode and LED's 14 will illuminate 15 the suspected tissue 10. Both these wavelength spectrums will cause fluorescence of the surrounding healthy tissue and permit differentiation of a suspected lesion 10. The operator 12 views the suspected lesions 10 through filter 2 and clearly sees its boundaries. This procedure may be carried out in a darkened room to increase contrast and eliminate ambient light. If more than one irradiating device 8 is used then one would be used to first examine in the range of between 340 to 370 nm and the other in the range between 420 nm to 450 nm. The filter 2 for the first irradiating device would preferably block light below 400-430 NM and the second would preferably block light below 480-510 NM. The examination would take place sequentially at the different excitation wavelengths.

The size of the filter 2 opening is very important. If the opening 17 is too large then the irradiation of light from the circular array of LED's will land far outside the oral cavity. If the opening 17 is too small it will limit the viewable area of a suspected lesion and increase the time it will take to perform the oral examination. If the opening 17 is very small, the procedure becomes difficult and impractical, since the lesion itself might be larger than the viewable opening. For these reasons a device for oral examination should have a minimum opening diameter of ½ inch and a maximum diameter of 2½ inches. Preferably the opening 17 should be 1 to 2 inches in diameter.

FIG. 3 shows a drive circuit 16 which controls the functions of the LED's. The circuit contains a voltage regulator 17, which is also a dc to dc converter to provide constant output to the LED's. For example, using two 1.5 volt batteries for the battery source B, a total of 3 volts is available. The voltage regulator 17 will maintain a constant output voltage to the LED's of, e.g., even if the total battery voltage drops to 2.5 volts, providing longer useful battery life. Switch 13 is a rocker switch with a center off position. By depressing the switch in one direction, LED array 1 is energized and in the opposite position, LED array 14 is energized. Alternatively, a drive circuit is shown in FIG. 4, could also include a microprocessor controller, 18 which determines which LED's go on and the sequence of control i.e. LED's-OFF, LED array 1-ON, LED array 14-ON. The microprocessor 18 would also control additional LED arrays should that be desired. It should be understood that although the filter 2 is preferably a high pass filter a band pass filter may be employed. Band pass filters are made by vacuum depositing many layers of metal oxide materials unto the glass. High and low pass filters are made by incorporating metal oxides into the glass during manufacturing process which is much less expensive. Because the LED source provides a very narrow defined spectrum of energy, a high pass filter which is a much lower cost and is less prone to degradation may be used in our invention.

1- An optical screening device for detecting abnormal skin tissue indicative of disease comprising:
a handle having an elongated stem adapted for holding the optical screening device in one hand and for placing the screening device into relatively close proximity to skin tissue for visual observation through the screening device;
a head connected to the elongated stem of the handle for housing an array of light emitting diodes with each diode in the array being spaced apart a predetermined distance to form an arrangement of more than at least three diodes in a given geometry with each diode selected to emit light in a wavelength range between 340 nm to 470 nm;
a high pass filter supported by said handle in close proximity to said array of light emitting diodes with the filter being of a size to provide a defined viewable area for visibly detecting irradiated light reflected from the skin tissue placed under observation by the screening device and adapted to block light at least below 380 nm; and a source of electrical power for energizing said array of light emitting diodes.

2- An optical screening device as defined in claim 1 wherein the skin tissue to be placed under observation is located in or about the oral cavity of the mouth.

3- An optical screening device as defined in claim 2 wherein said the said array of light emitting diodes surrounds said high pass filter such that the defined viewable area is substantially circumscribed by said array of light emitting diodes.

4- An optical screening device as defined in claim 3 wherein the geometry formed by said array of light emitting diodes is circular and said viewable area is limited to about 2½ inches in diameter.

5- An optical screening device as defined in claim 4 wherein said array of light emitting diodes emits light in the 340 nm to 370 nm range.

6- An optical screening device as defined in claim 5 wherein said high pass filter blocks light below 370 nm.

7- An optical screening device as defined in claim 4 wherein said array of light emitting diodes emits light in the 420 nm to 450 nm range.

8- An optical screening device as defined in claim 7 wherein said high pass filter blocks light below 450 nm.

9- An optical screening device as defined in claim 2 wherein said array of light emitting diodes includes a first plurality of light emitting diodes for emitting light in a selected first wavelength range of between 340 nm to 2370 nm and a second plurality of light emitting diodes for emitting light in a selected second wavelength range of between 420 nm to 450 nm.

10- An optical screening device as defined in claim 9 wherein said first plurality of light emitting diodes and said second plurality of light emitting diodes are arranged to substantially form concentric circles with said high pass filter surrounded by both said first and second plurality of light emitting diodes.

11- An optical screening device as defined in claim 10 further comprising switching means for selectively connecting said power source to said first or second plurality of light emitting diodes.

12- An optical screening device as defined in claim 11 wherein said switching means is a rocker switch.

13- An optical screening device as defined in claim 10 further comprising a drive circuit for energizing said first plurality of first and said second light emitting diodes from said source of power, switching means for selectively connecting said power source to either said first or second plurality of light emitting diodes and a microprocessor for providing a controlled sequence for activating said drive circuit.

14- An optical screening device as defined in claim 13 wherein said switching means is a push button switch.
